(19) United States
(12) Patent Application Publication
(10) Pub. No.: US 2006/0199202 A1
(43) Pub. Date: Sep. 7, 2006

(54) Detection of allelic expression imbalance

(75) Inventors: Victor Lyamichev, Madison, WI (US); Hatim Allawi, Madison, WI (US)

Correspondence Address: MEDLEN & CARROLL, LLP, 101 HOWARD STREET, SUITE 350, SAN FRANCISCO, CA, 94105, US

(73) Assignee: Third Wave Technologies, Inc., Madison, WI (US)

(21) Appl. No.: 11/349,872

(22) Filed: Feb. 8, 2006

Related U.S. Application Data
(60) Provisional application No. 60/651,408, filed on Feb. 9, 2005.

(51) Int. Cl. C12Q 1/68

(52) U.S. Cl. 435/6

(57) ABSTRACT

The present invention provides compositions and methods for the detection and characterization of allelic expression imbalance from a heterozygous gene locus. More particularly, the present invention provides compositions, kits, and methods for the determination of allelic expression imbalance by the comparison of expression levels from each of two alleles of a given gene locus through the use of an invasive cleavage structure assay (e.g. the INVADER assay).

The present Application claims priority to U.S. Provisional Application Ser. No. 60/651,408, filed Feb. 9, 2005, which is herein incorporated by reference.

### FIELD OF THE INVENTION

The present invention provides compositions, kits, and methods for the detection and characterization of allelic expression imbalance from a heterozygous gene locus. More particularly, the present invention provides compositions, kits, and methods for the determination of allelic expression imbalance by the comparison of expression levels from each of two alleles of a given gene locus through the use of an invasive cleavage structure assay (e.g. the INVADER assay).

### BACKGROUND

Single nucleotide polymorphisms (SNPs) are highly abundant in the human genome, appearing on average at 0.1% of the nucleotide positions. Thus, each gene or transcriptional unit will likely contain multiple SNPs that potentially give rise to sequence variation between individuals and tissues on the level of RNA. Recent studies indicate that differences in the expression levels of the alleles of heterozygous SNPs (allelic expression imbalance) may occur frequently for human genes. Non-synonymous SNPs in coding regions of genes may be functional by altering an amino acid, which in turn may affect the structure and function of the encoded protein, while synonymous SNPs may have functional consequences by affecting the stability or folding of mRNA transcripts. Intronic SNPs may give rise to alternatively spliced mRNAs, while SNPs in 5′- or 3′-untranslated mRNA regions may affect the stability or processing of the RNA. Moreover, SNPs in non-protein coding regions of genes that affect binding of regulatory factors may cause imbalanced expression of SNP alleles. This form of genetic variation has been suggested as a common cause of both normal and disease-related inter-individual variation in complex phenotypes. (See, e.g., Yan and Zhou, Curr. Opin. Oncol., 16:39-43, 2004; and Hudson T J. Nat Genet 33:439-440, 2003; both of which are herein incorporated by reference in their entireties.)

Epigenetic mechanisms involved in the establishment of allelic exclusion play a central role in many types of monoallelic expression, including lymphocyte maturation, X-chromosome inactivation in female cells, and parental imprinting. In all three systems, the inequality of the two alleles seems to be achieved mainly by differential DNA methylation, asynchronous DNA replication, differential chromatin modifications, unequal nuclear localization, and non-coding RNA (See, e.g., Goldmit and Bergman, Immunol. Rev. 200:197-214, 2004, herein incorporated by reference in its entirety).

Certain methods for the detection of imbalanced expression of two alleles in a heterozygote are known in the art. For example, minisequencing methods may be employed as described in Liljedhal, U., et. al. BCM Biotechnology 4:24, 2004, which is herein incorporated by reference in its entirety. Existing techniques for detection of allelic expression imbalance possess inherent limitations such as high rates of false positive and negative detection, limited quantitation dynamic range, high cost, long time periods for results, and requirements for large quantities of target material in the specimen. Therefore, there exists a need for a rapid, sensitive, and highly quantitative direct detection assay for detecting imbalanced allelic expression.

### SUMMARY OF THE INVENTION

The present invention provides compositions, kits, and methods for the detection and characterization of allelic expression imbalance from a heterozygous gene locus. More particularly, the present invention provides compositions, kits, and methods for the determination of allelic expression imbalance by the comparison of expression levels from each of two alleles of a given gene locus through the use of an invasive cleavage structure assay (e.g. the INVADER assay).

In certain embodiments, the present invention provides methods of detecting the presence or absence of allelic expression imbalance from a heterozygous gene locus, comprising; a) providing a sample comprising a population of target nucleic acid sequences, wherein the target nucleic acid sequences comprise: i) mRNA transcripts produced from the heterozygous gene locus, ii) cDNA products produced from the mRNA transcripts; or iii) amplified products produced from the cDNA products; b) contacting the sample with invasive cleavage assays (e.g. INVADER assays) under conditions such that a quantitative signal for a first allele and a second allele in the population of target nucleic acid sequences is determined; and c) comparing the quantitative signal for the first and second alleles to determine the presence or absence of allelic expression imbalance from the heterozygous gene locus in the sample.

In some embodiments, the present invention provides methods of detecting the presence or absence of allelic expression imbalance from a heterozygous gene locus, comprising; a) providing a sample comprising; i) a first nucleic acid population comprising target nucleic acid molecules selected from: i) genomic DNA molecules comprising the heterozygous gene locus, or ii) a first amplified product produced from the genomic DNA molecules; and ii) a second nucleic acid population comprising target nucleic acid molecules selected from: i) mRNA transcripts produced from the heterozygous gene locus, ii) cDNA products produced from the mRNA transcripts; or iii) a second amplified product produced from the cDNA products; b) contacting the sample with invasive cleavage assays (e.g. INVADER assays) under conditions such that a quantitative signal for a first allele and a second allele in the first and in the second nucleic acid populations is determined; and c) comparing the quantitative signal for the first and second alleles in the second nucleic acid population to each other and to the quantitative signal for the first and second alleles in the first nucleic acid population to determine the presence or absence of allelic expression imbalance from the heterozygous gene locus in the sample.

In some embodiments, the invasive cleavage assays comprise INVADER assay reagents. In particular embodiments, the invasive cleavage assays comprise first and second oligonucleotides, wherein the first and second oligonucleotides are configured to form invasive cleavage structures with the target nucleic acid sequences. In some embodiments, the first oligonucleotides comprise a 5′ portion and a 3′ portion, wherein the 3′ portion is configured to hybridize to the target nucleic acid sequences, and wherein the 5′ portion is configured to not hybridize to the target nucleic acid sequences. In additional embodiments, the second oligonucleotides comprise a 5′ portion and a 3′ portion, wherein the 5′ portion is configured to hybridize to the target nucleic acid sequences, and wherein the 3′ portion is configured to not hybridize to the target nucleic acid sequences.

In some embodiments, the sample is a biological sample from a subject (e.g. blood sample, semen sample, urine sample, biopsy sample, etc.). In particular embodiments, the methods further comprise step d) identifying the subject as having a particular condition based on the presence or absence of allelic expression imbalance from the heterzygous gene locus in the sample. In further embodiments, the particular condition is selected from the group consisting of: cancer (e.g., breast cancer, brain cancer, pancreatic cancer), loss of heterozygosity, loss of imprinting, proper imprinting, Prader-Willi syndrome, Angelman syndrome, Beckwith-Wiedmann syndrome, Silver-Russel syndrome, diabetes, gestational diabetes, autism, bipolar affective disorder, epilepsy, schizophrenia, Tourette syndrome and Turner syndrome.

In certain embodiments, the quantitative signal from the first allele is at least two percent greater than the quantitative signal from the second allele (e.g. 51/49, or 55/45, or 60/40, or 70/30, etc.), and wherein the comparing determines the presence of allelic expression imbalance from the heterozygous gene locus in the sample. In particular embodiments, the quantitative signal from the first allele is about the same as the quantitative signal from the second allele (e.g. 50.5/49.5 or 50/50), and the comparing determine the absence of allelic expression imbalance. In some embodiments, the quantitative signals from said first and second alleles are distinct from each other. In preferred embodiments, the invasive cleavage assays are configured to detect single nucleotide polymorphisms in the target nucleic acid sequences in order to generate the quantitative signal for the first allele and the second allele. In some embodiments, the amplified products are produced from the genomic DNA or cDNA products via polymerase chain reaction or other amplification method.

In particular embodiments, the present invention provides kits comprising first and second invasive cleavage assays configured for detecting the presence or absence of allelic expression imbalance from a heterozygous gene locus, wherein the first invasive cleavage assay is configured to generate a quantitative signal for a first allele of the heterozygous gene locus, and the second invasive cleavage assay is configured to generate a quantitative signal for a second allele of the heterozygous gene locus. In certain embodiments, the first and second invasive cleavage assays comprise INVADER assay reagents. In some embodiments, the first and second invasive cleavage assays comprise first and second oligonucleotides, wherein the first and second oligonucleotides are configured to form invasive cleavage structures with target nucleic acid sequences. In additional embodiments, the first oligonucleotides comprise a 5′ portion and a 3′ portion, wherein the 3′ portion is configured to hybridize to the target nucleic acid sequences, and wherein the 5′ portion is configured to not hybridize to the target nucleic acid sequences.

Definitions

To facilitate an understanding of the present invention, a number of terms and phrases are defined below:

As used herein, the terms “subject” and “patient” refer to any organisms including plants, microorganisms and animals (e.g., mammals such as dogs, cats, livestock, and humans).

As used herein, the term “INVADER assay reagents” refers to one or more reagents for detecting target sequences, said reagents comprising oligonucleotides capable of forming an invasive cleavage structure in the presence of the target sequence. In some embodiments, the INVADER assay reagents further comprise an agent for detecting the presence of an invasive cleavage structure (e.g., a cleavage agent). In some embodiments, the oligonucleotides comprise first and second oligonucleotides, said first oligonucleotide comprising a 5′ portion complementary to a first region of the target nucleic acid and said second oligonucleotide comprising a 3′ portion and a 5′ portion, said 5′ portion complementary to a second region of the target nucleic acid downstream of and contiguous to the first portion. In some embodiments, the 3′ portion of the second oligonucleotide comprises a 3′ terminal nucleotide not complementary to the target nucleic acid. In preferred embodiments, the 3′ portion of the second oligonucleotide consists of a single nucleotide not complementary to the target nucleic acid.

In some embodiments, INVADER assay reagents are configured to detect a target nucleic acid sequence comprising first and second non-contiguous single-stranded regions separated by an intervening region comprising a double-stranded region. In preferred embodiments, the INVADER assay reagents comprise a bridging oligonucleotide capable of binding to said first and second non-contiguous single-stranded regions of a target nucleic acid sequence. In particularly preferred embodiments, either or both of said first or said second oligonucleotides of said INVADER assay reagents are bridging oligonucleotides.

In some embodiments, the INVADER assay reagents further comprise a solid support. For example, in some embodiments, the one or more oligonucleotides of the assay reagents (e.g., first and/or second oligonucleotide, whether bridging or non-bridging) is attached to said solid support. In some embodiments, the INVADER assay reagents further comprise a buffer solution. In some preferred embodiments, the buffer solution comprises a source of divalent cations (e.g., Mn²⁺ and/or Mg²⁺ ions). Individual ingredients (e.g., oligonucleotides, enzymes, buffers, target nucleic acids) that collectively make up INVADER assay reagents are termed “INVADER assay reagent components.”

In some embodiments, the INVADER assay reagents further comprise a third oligonucleotide complementary to a third portion of the target nucleic acid upstream of the first portion of the first target nucleic acid. In yet other embodiments, the INVADER assay reagents further comprise a target nucleic acid. In some embodiments, the INVADER assay reagents further comprise a second target nucleic acid. In yet other embodiments, the INVADER assay reagents further comprise a third oligonucleotide comprising a 5′ portion complementary to a first region of the second target nucleic acid. In some specific embodiments, the 3′ portion of the third oligonucleotide is covalently linked to the second target nucleic acid. In other specific embodiments, the second target nucleic acid further comprises a 5′ portion, wherein the 5′ portion of the second target nucleic acid is the third oligonucleotide. In still other embodiments, the INVADER assay reagents further comprise an ARRESTOR molecule (e.g., ARRESTOR oligonucleotide).

In some preferred embodiments, the INVADER assay reagents further comprise reagents for detecting a nucleic acid cleavage product. In some embodiments, one or more oligonucleotides in the INVADER assay reagents comprise a label. In some preferred embodiments, said first oligonucleotide comprises a label. In other preferred embodiments, said third oligonucleotide comprises a label. In particularly preferred embodiments, the reagents comprise a first and/or a third oligonucleotide labeled with moieties that produce a fluorescence resonance energy transfer (FRET) effect.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In preferred embodiments, predispensed assay reagent components are predispensed and are provided in a reaction vessel (including but not limited to a reaction tube or a well, as in, e.g., a microtiter plate). In certain preferred embodiments, the INVADER assay reagents are provided in microfluidic devices such as those described in U.S. Pat. Nos. 6,627,159; 6,720,187; 6,734,401; and 6,814,935, as well as U.S. Pat. Pub. 2002/0064885, all of which are herein incorporated by reference. In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides.

In some embodiments, the present invention provides INVADER assay reagent kits comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, software (e.g., for collecting and analyzing data), inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; mass tags; and fluorescent dyes alone or in combination with moieties that can suppress (“quench”) or shift emission spectra by fluorescence resonance energy transfer (FRET). FRET is a distance-dependent interaction between the electronic excited states of two molecules (e.g., two dye molecules, or a dye molecule and a non-fluorescing quencher molecule) in which excitation is transferred from a donor molecule to an acceptor molecule without emission of a photon. In some embodiments, changes in detectable emission from a donor dye (e.g. when an acceptor moiety is near or distant) are detected. In other embodiments, changes in detectable emission from an acceptor dye are detected. In preferred embodiments, the emission spectrum of the acceptor dye is distinct from the emission spectrum of the donor dye, such that the signals can be differentiated from each other.

In some embodiments, a donor dye is used in combination with multiple acceptor moieties. In a preferred embodiment, a donor dye is used in combination with a non-fluorescing quencher and with an acceptor dye, such that when the donor dye is close to the quencher, its excitation is transferred to the quencher rather than the acceptor dye, and when the quencher is removed (e.g., by cleavage of a probe), donor dye excitation is transferred to an acceptor dye. In particularly preferred embodiments, emission from the acceptor dye is detected. See, e.g., Tyagi, et al., Nature Biotechnology 18:1191 (2000), which is incorporated herein by reference.

Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “T,” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of Tₘ.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The term “gene locus” refers the place on a chromosome where a gene is located. An individual may have two different alleles of a gene at a given locus (e.g. one allele of the gene on the maternal chromosome and a different allele of the gene on the paternal chromosome, and therefore is heterozygous at this gene locus).

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant” or “polymorphic” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof. In some embodiments, oligonucleotides that form invasive cleavage structures are generated in a reaction (e.g., by extension of a primer in an enzymatic extension reaction).

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′end” if its 5′ phosphate is not linked to the 3′ oxygen of a mononucleotide pentose ring and as the “3′end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “cleavage structure” as used herein, refers to a structure that is formed by the interaction of at least one probe oligonucleotide and a target nucleic acid, forming a structure comprising a duplex, the resulting structure being cleavable by a cleavage means, including but not limited to an enzyme. The cleavage structure is a substrate for specific cleavage by the cleavage means in contrast to a nucleic acid molecule that is a substrate for non-specific cleavage by agents such as phosphodiesterases which cleave nucleic acid molecules without regard to secondary structure (i.e., no formation of a duplexed structure is required).

The term “cleavage means” or “cleavage agent” as used herein refers to any means that is capable of cleaving a cleavage structure, including but not limited to enzymes. “Structure-specific nucleases” or “structure-specific enzymes” are enzymes that recognize specific secondary structures in a nucleic molecule and cleave these structures. The cleavage means of the invention cleave a nucleic acid molecule in response to the formation of cleavage structures; it is not necessary that the cleavage means cleave the cleavage structure at any particular location within the cleavage structure.

The cleavage means may include nuclease activity provided from a variety of sources including the CLEAVASE enzymes, the FEN-1 endonucleases (including RAD2 and XPG proteins), Taq DNA polymerase and E. coli DNA polymerase I. The cleavage means may include enzymes having 5′ nuclease activity (e.g., Taq DNA polymerase (DNAP), E. coli DNA polymerase I). The cleavage means may also include modified DNA polymerases having 5′ nuclease activity but lacking synthetic activity. Examples of cleavage means suitable for use in the method and kits of the present invention are provided in U.S. Pat. Nos. 5,614,402; 5,795,763; 5,843,669; 6,090; PCT Appln. Nos WO 98/23774; WO 02/070755A2; and WO0190337A2, each of which is herein incorporated by reference it its entirety.

The term “thermostable” when used in reference to an enzyme, such as a 5′ nuclease, indicates that the enzyme is functional or active (i.e., can perform catalysis) at an elevated temperature, i.e., at about 55° C. or higher.

The term “cleavage products” as used herein, refers to products generated by the reaction of a cleavage means with a cleavage structure (i.e., the treatment of a cleavage structure with a cleavage means).

The term “target nucleic acid,” when used in reference to an invasive cleavage reaction, refers to a nucleic acid molecule containing a sequence that has at least partial complementarity with at least a probe oligonucleotide and may also have at least partial complementarity with an INVADER oligonucleotide. The target nucleic acid may comprise single- or double-stranded DNA or RNA.

The term “non-target cleavage product” refers to a product of a cleavage reaction that is not derived from the target nucleic acid. As discussed above, in the methods of the present invention, cleavage of the cleavage structure generally occurs within the probe oligonucleotide. The fragments of the probe oligonucleotide generated by this target nucleic acid-dependent cleavage are “non-target cleavage products.”

The term “probe oligonucleotide,” when used in reference to an invasive cleavage reaction, refers to an oligonucleotide that interacts with a target nucleic acid to form a cleavage structure in the presence or absence of an INVADER oligonucleotide. When annealed to the target nucleic acid, the probe oligonucleotide and target form a cleavage structure and cleavage occurs within the probe oligonucleotide.

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide-whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide.

The term “cassette,” when used in reference to an invasive cleavage reaction, as used herein refers to an oligonucleotide or combination of oligonucleotides configured to generate a detectable signal in response to cleavage of a probe oligonucleotide in an INVADER assay. In preferred embodiments, the cassette hybridizes to a non-target cleavage product from cleavage of the probe oligonucleotide to form a second invasive cleavage structure, such that the cassette can then be cleaved.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label. In particularly preferred embodiments, cassette comprises labeled moieties that produce a fluorescence resonance energy transfer (FRET) effect.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The term “liberating” as used herein refers to the release of a nucleic acid fragment from a larger nucleic acid fragment, such as an oligonucleotide, by the action of, for example, a 5′ nuclease such that the released fragment is no longer covalently attached to the remainder of the oligonucleotide.

The term “Kₘ” as used herein refers to the Michaelis-Menten constant for an enzyme and is defined as the concentration of the specific substrate at which a given enzyme yields one-half its maximum velocity in an enzyme catalyzed reaction.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include comprise modified forms of deoxyribonucleotides as well as ribonucleotides. The nucleic acid components of the invasive cleavage assays may comprise one or more nucleotide analogs.

The term “polymorphic locus” is a locus present in a population that shows variation between members of the population (e.g., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The term “source of target nucleic acid” refers to any sample that contains nucleic acids (RNA or DNA). Particularly preferred sources of target nucleic acids are biological samples including, but not limited to blood, saliva, cerebral spinal fluid, pleural fluid, milk, lymph, sputum and semen.

An oligonucleotide is said to be present in “excess” relative to another oligonucleotide (or target nucleic acid sequence) if that oligonucleotide is present at a higher molar concentration that the other oligonucleotide (or target nucleic acid sequence). When an oligonucleotide such as a probe oligonucleotide is present in a cleavage reaction in excess relative to the concentration of the complementary target nucleic acid sequence, the reaction may be used to indicate the amount of the target nucleic acid present. Typically, when present in excess, the probe oligonucleotide will be present at least a 100-fold molar excess; typically at least 1 pmole of each probe oligonucleotide would be used when the target nucleic acid sequence was present at about 10 fmoles or less.

The term “nucleic acid sequence” as used herein refers to an oligonucleotide, nucleotide or polynucleotide, and fragments or portions thereof, and to DNA or RNA of genomic or synthetic origin that may be single or double stranded, and represent the sense or antisense strand. Similarly, “amino acid sequence” as used herein refers to peptide or protein sequence.

### DESCRIPTION OF THE DRAWING

FIG. 1 shows a schematic diagram of INVADER oligonucleotides, probe oligonucleotides and FRET cassettes for detecting a wild-type single-nucleotide polymorphism.

### DESCRIPTION OF THE INVENTION

The present invention provides compositions, kits, and methods for the detection and characterization of allelic expression imbalance from a heterozygous gene locus. More particularly, the present invention provides compositions, kits, and methods for the determination of allelic expression imbalance by the comparison of expression levels from each of two alleles of a given gene locus through the use of an invasive cleavage structure assay (e.g. the INVADER assay).

I. Invasive Cleavage Assays

The present invention provides means for forming a nucleic acid cleavage structure that is dependent upon the presence of a target nucleic acid and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced. Such embodiments have been termed the INVADER assay (Third Wave Technologies) and are described in U.S. Pat. Nos. 5,846,717, 5,985,557, 5,994,069, 6,001,567, and 6,090,543, WO 97/27214 WO 98/42873, Lyamichev et al., Nat. Biotech., 17:292 (1999), Hall et al., PNAS, USA, 97:8272 (2000), each of which is herein incorporated by reference in their entirety for all purposes). The INVADER assay detects hybridization of probes to a target by enzymatic cleavage of specific structures by structure specific enzymes.

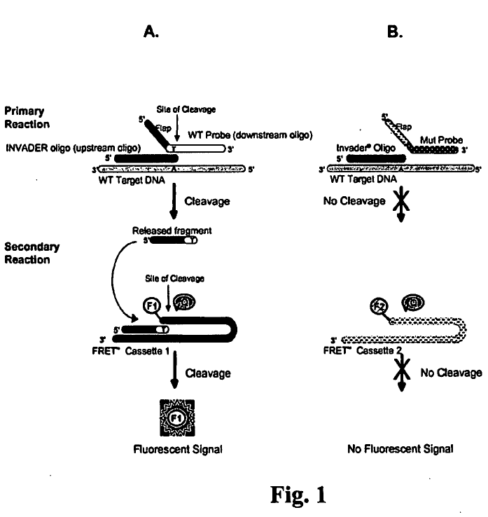

The INVADER assay detects specific DNA and RNA sequences by using structure-specific enzymes (e.g. FEN endonucleases) to cleave a complex formed by the hybridization of overlapping oligonucleotide probes (See, e.g. FIG. 1). Elevated temperature and an excess of one of the probes enable multiple probes to be cleaved for each target sequence present without temperature cycling. In some embodiments, these cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified, as well as amplified, RNA and DNA including genomic DNA. In the embodiments shown schematically in FIG. 1, the INVADER assay uses two cascading steps (a primary and a secondary reaction) both to generate and then to amplify the target-specific signal. For convenience, the alleles in the following discussion are described as wild-type (WT) and mutant (MT), even though this terminology does not apply to all genetic variations. In the primary reaction (FIG. 1, panel A), the WT primary probe and the INVADER oligonucleotide hybridize in tandem to the target nucleic acid to form an overlapping structure. An unpaired “flap” is included on the 5′ end of the WT primary probe. A structure-specific enzyme (e.g. the CLEAVASE enzyme, Third Wave Technologies) recognizes the overlap and cleaves off the unpaired flap, releasing it as a target-specific product. In the secondary reaction, this cleaved product serves as an INVADER oligonucleotide on the WT fluorescence resonance energy transfer (WT-FRET) probe to again create the structure recognized by the structure specific enzyme (panel A). When the two dyes on a single FRET probe are separated by cleavage (indicated by the arrow in FIG. 1), a detectable fluorescent signal above background fluorescence is produced. Consequently, cleavage of this second structure results in an increase in fluorescence, indicating the presence of the WT allele (or mutant allele if the assay is configured for the mutant allele to generate the detectable signal). In preferred embodiments, FRET probes having different labels (e.g. resolvable by difference in emission or excitation wavelengths, or resolvable by time-resolved fluorescence detection) are provided for each allele or locus to be detected, such that the different alleles or loci can be detected in a single reaction. In such embodiments, the primary probe sets and the different FRET probes may be combined in a single assay, allowing comparison of the signals from each allele or locus in the same sample.

If the primary probe oligonucleotide and the target nucleotide sequence do not match perfectly at the cleavage site (e.g., as with the MT primary probe and the WT target, FIG. 1, panel B), the overlapped structure does not form and cleavage is suppressed. The structure specific enzyme (e.g., CLEAVASE VIII enzyme, Third Wave Technologies) used cleaves the overlapped structure more efficiently (e.g. at least 340-fold) than the non-overlapping structure, allowing excellent discrimination of the alleles.

In the INVADER assays, the probes turn can over without temperature cycling to produce many signals per target (i.e., linear signal amplification). Similarly, each target-specific product can enable the cleavage of many FRET probes.

The primary INVADER assay reaction is directed against the target DNA (or RNA) being detected. The target DNA is the limiting component in the first invasive cleavage, since the INVADER and primary probe are supplied in molar excess. In the second invasive cleavage, it is the released flap that is limiting. When these two cleavage reactions are performed sequentially, the fluorescence signal from the composite reaction accumulates linearly with respect to the target DNA amount.

In certain embodiments, the INVADER assay, or other nucleotide detection assays, are performed with accessible site designed oligonucleotides and/or bridging oligonucleotides. Such methods, procedures and compositions are described in U.S. Pat. No. 6,194,149, WO9850403, and WO0198537, all of which are specifically incorporated by reference in their entireties.

In certain embodiments, the target nucleic acid sequences are amplified prior to detection (e.g. such that amplified products are generated). In some embodiments, the target nucleic acid comprises genomic DNA. In other embodiments, the target nucleic acid comprises synthetic DNA or RNA. In some preferred embodiments, synthetic DNA within a sample is created using a purified polymerase. In some preferred embodiments, creation of synthetic DNA using a purified polymerase comprises the use of PCR. In other preferred embodiments, creation of synthetic DNA using a purified DNA polymerase, suitable for use with the methods of the present invention, comprises use of rolling circle amplification, (e.g., as in U.S. Pat. Nos. 6,210,884, 6,183,960 and 6,235,502, herein incorporated by reference in their entireties). In other preferred embodiments, creation of synthetic DNA comprises copying genomic DNA by priming from a plurality of sites on a genomic DNA sample. In some embodiments, priming from a plurality of sites on a genomic DNA sample comprises using short (e.g., fewer than about 8 nucleotides) oligonucleotide primers. In other embodiments, priming from a plurality of sites on a genomic DNA comprises extension of 3′ ends in nicked, double-stranded genomic DNA (i.e., where a 3′ hydroxyl group has been made available for extension by breakage or cleavage of one strand of a double stranded region of DNA). Some examples of making synthetic DNA using a purified polymerase on nicked genomic DNAs, suitable for use with the methods and compositions of the present invention, are provided in U.S. Pat. No. 6,117,634, issued Sep. 12, 2000, and U.S. Pat. No. 6,197,557, issued Mar. 6, 2001, and in PCT application WO 98/39485, each incorporated by reference herein in their entireties for all purposes.

In some embodiments, the present invention provides methods for detecting a target sequence, comprising: providing a) a sample containing DNA amplified by extension of 3′ ends in nicked double-stranded genomic DNA, said genomic DNA suspected of containing said target sequence; b) oligonucleotides capable of forming an invasive cleavage structure in the presence of said target sequence; and c) exposing the sample to the oligonucleotides and the agent. In some embodiments, the agent comprises a cleavage agent. In some particularly preferred embodiments, the method of the invention further comprises the step of detecting said cleavage product.

In some preferred embodiments, the exposing of the sample to the oligonucleotides and the agent comprises exposing the sample to the oligonucleotides and the agent under conditions wherein an invasive cleavage structure is formed between said target sequences and said oligonucleotides if said target sequences are present in said sample, wherein said invasive cleavage structure is cleaved by said cleavage agent to form a cleavage product.

In some particularly preferred embodiments, the target sequence comprises a first region and a second region, said second region downstream of and contiguous to said first region, and said oligonucleotides comprise first and second oligonucleotides, said wherein at least a portion of said first oligonucleotide is completely complementary to said first portion of said target sequence and wherein said second oligonucleotide comprises a 3′ portion and a 5′ portion, wherein said 5′ portion is completely complementary to said second portion of said target nucleic acid.

In other embodiments, synthetic DNA suitable for use with the methods and compositions of the present invention is made using a purified polymerase on multiply-primed genomic DNA, as provided, e.g., in U.S. Pat. Nos. 6,291,187, and 6,323,009, and in PCT applications WO 01/88190 and WO 02/00934, each herein incorporated by reference in their entireties for all purposes. In these embodiments, amplification of DNA such as genomic DNA is accomplished using a DNA polymerase, such as the highly processive Φ 29 polymerase (as described, e.g., in U.S. Pat. Nos. 5,198,543 and 5,001,050, each herein incorporated by reference in their entireties for all purposes) in combination with exonuclease-resistant random primers, such as hexamers.

In some embodiments, the present invention provides methods for detecting a target sequence, comprising: providing a) a sample containing DNA amplified by extension of multiple primers on genomic DNA, said genomic DNA suspected of containing said target sequence; b) oligonucleotides capable of forming an invasive cleavage structure in the presence of said target sequence; and c) exposing the sample to the oligonucleotides and the agent. In some embodiments, the agent comprises a cleavage agent. In some preferred embodiments, said primers are random primers. In particularly preferred embodiments, said primers are exonuclease resistant. In some particularly preferred embodiments, the method of the invention further comprises the step of detecting said cleavage product.

In some preferred embodiments, the exposing of the sample to the oligonucleotides and the agent comprises exposing the sample to the oligonucleotides and the agent under conditions wherein an invasive cleavage structure is formed between said target sequence and said oligonucleotides if said target sequence is present in said sample, wherein said invasive cleavage structure is cleaved by said cleavage agent to form a cleavage product.

In some particularly preferred embodiments, the target sequence comprises a first region and a second region, said second region downstream of and contiguous to said first region, and said oligonucleotides comprise first and second oligonucleotides, said wherein at least a portion of said first oligonucleotide is completely complementary to said first portion of said target sequence and wherein said second oligonucleotide comprises a 3′ portion and a 5′ portion, wherein said 5′ portion is completely complementary to said second portion of said target nucleic acid.

The present invention further provides assays in which the target nucleic acid is reused or recycled during multiple rounds of hybridization with oligonucleotide probes and cleavage of the probes without the need to use temperature cycling (e.g., for periodic denaturation of target nucleic acid strands) or nucleic acid synthesis (e.g., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (The kinetics of oligonucleotide replacement. Luis P. Reynaldo, Alexander V. Vologodskii, Bruce P. Neri and Victor I. Lyamichev. J. Mol. Biol. 97: 511-520 (2000)), multiple probes can hybridize to the same target, allowing multiple cleavages, and the generation of multiple cleavage products.

II. RNA Detection via Invasive Cleavage Assays

As decribed above for the detection of multiple alleles, multiplex formats of the RNA INVADER assay enable simultaneous expression analysis of two or more genes within the same sample. In a primary reaction, one-nucleotide overlap-substrates are generated by the hybridization of INVADER oligonucleotides and probe oligonucleotides to their respective RNA targets (e.g. mRNA from two alleles of the same gene locus). Each probe contains a specific, target-complementary region and a distinctive non-complementary 5′ flap that is associated only with that specific mRNA in that assay. The distinctive flaps may be distinguished in any of the myriad ways disclosed herein (e.g., with different labels, different secondary cleavage systems having different labels, specific antibodies, different sizes when resolved, differenct sequences detected by hybridization in solution or on surfaces, etc.)

While the RNA invasive cleavage assay, like the method used for DNA detection described above, can use two invasive cleavage reactions in sequence, its preference for the 5′ nucleases derived from DNA polymerases indicates that additional format changes are preferred. Unlike the FEN 5′ nucleases generally used for detection of DNA targets, optimal signal amplification with the DNA Pol-related 5′ nucleases occurs only when a probe turnover mechanism is employed in both the primary and secondary reactions (in contrast to an INVADER oligonucleotide turnover mechanism, wherein an INVADER oligonucleotide cycles, e.g., to direct the cleavage of multiple FRET cassettes). Consequently, in certain embodiments, RNA detection uses sequential operation of the two reactions, rather than simultaneous reaction performance. Because the reactions are performed truly sequentially, in these embodiments, the RNA INVADER assay signal accumulates linearly in both a target- and time-dependent manner. In contrast, the primary and secondary reactions of the DNA INVADER assay, when run concurrently, amplify signal as a linear function of target level, but as a quadratic function of time. In the sequential embodiments, the RNA INVADER assay uses two separate oligonucleotides, a secondary probe (e.g., a FRET probe) and secondary target, for signal generation.

A feature of the RNA invasive cleavage assay is its ability to discriminate highly homologous RNA sequences, such as those from two different alleles of the same gene locus that only differ by a single base. Like the DNA INVADER assay, the RNA INVADER assay can discriminate single-base changes. In some embodiments, the first 5′ complementary base of each probe is positioned at a non-conserved site in its mRNA target, so that a mismatch prevents formation of the overlap-structure, and thus prevents cleavage of the probe. Alternatively spliced mRNA variants can be specifically detected by positioning the cleavage site at a splice junction.

To monitor large changes in mRNA levels, the dynamic range of the assay can be extended using real-time analysis. However, since the assay generates signal linearly with time or target level, simply varying the amount of sample added per reaction and calculating the copies of mRNA per ng total RNA enables accurate quantitation with a single endpoint measurement on low-cost instrumentation. Further, in cases where absolute quantitation is not necessary, the assay's linear signal amplification mechanism and reproducibility also eliminate the need for a standard curve and enable simple and precise relative quantitation of any one gene.

The RNA INVADER assay is particularly suited for detecting alternatively spliced or edited RNA variants because even a single base change at the overlap site affects 5′ nuclease cleavage. Splice variants can be monitored in at least two ways with the assay: 1) detection of an individual exon or 2) detection of a specific splice junction.

To examine an RNA population for variants having more or fewer exons after splicing, INVADER assay probe sets are designed for each of the exons of interest (or for all exons in the mature RNA). Quantitation of exons, independent of how many mRNAs they reside in, may provide information about the number of splice variants for a given gene, as well as indicate the levels of expression for each exon. Mini in vitro transcripts containing only one or a few exons can be generated for each probe set so that absolute quantitation can be performed for each exon, thus enabling accurate comparisons of exon levels. If it is known that a particular exon is present in all known variants, in some embodiments, a probe set is designed for that exon for use as an internal control to normalize across different samples. RNAs having a one copy of each exon (e.g., “normally spliced” RNA) should produce signal from the collection of probe sets in certain relative amounts (which should be essentially equal for all exons, corrected for variations in the sensitivity of individual probe sets). Alterations in splicing alter the relative amounts of the exons. For example, if all of the produced RNAs are missing one of the normal exons, the signal for that exon drops toward zero, while if half of the RNAs are missing that exon, the signal for that exon drops toward 50%. More complex combinations of splice variations and mixtures of differently spliced mRNAs yield more complex and more informative profiles. Detection is not limited to exons. RNA populations may also be monitored for the presence of intron sequences that are usually removed by splicing.

Additionally, in some embodiments, the mRNA INVADER assay is also used to monitor alternative start and stop sites in the mRNA, and is used to monitor lifetimes of processed and unprocessed RNAs and RNA fragments (e.g., as used in timecourse studies following induction).

Approaches to designing INVADER assays for the detection of RNA targets can vary depending on the needs of a particular assay. For example, in some embodiments, an RNA to be detected or analyzed may be present in a test sample at low levels, so a high level of sensitivity (e.g., a low limit of detection, or LOD) may be desirable; in other embodiments, an RNA may abundant, and may not require an especially sensitive assay for detection. In some embodiments, an RNA to be detected may be similar to other RNAs in a sample that are not intended to be detected, so that a high level of selectivity in an assay is desirable, while in other embodiments, it may be desired that multiple similar RNAs be detected in a single reaction, so an assay may be provided that is not selective with respect to the differences among these similar RNAs.

In some embodiments it is desirable to avoid detection of any DNA molecules related to the target RNA molecules in a reaction. In some embodiments, this is accomplished by designing INVADER assay probe sets to RNA splice junctions, such that only the properly spliced mRNAs provide the selected target sites for detection. In other embodiments, samples are handled such that DNA remains double stranded (e.g., the nucleic acids are not heated or otherwise subjected to denaturing conditions), and is thus not available to serve as target in an INVADER assay reaction. In other embodiments, cells are lysed under conditions that leave nuclei intact, thereby containing and preventing detection of the genomic DNA, while releasing the cytosolic mRNAs into the lysate solution for detection by the assay.

In some embodiments, the INVADER assay is to be used for detection or quantitation of an entire RNA having a particular variation of a sequence (e.g., a SNP, a particular spliced junction, etc.); in such embodiments, the location of the base or sequence to be detected is a determining factor in the selection of a site for the INVADER assay probe set to hybridize. In other embodiments, any portion of an RNA target may be used to indicate the presence or the amount of the entire RNA (e.g., as in gene expression analysis). In this case, the probe sets may be directed toward a portion of the RNA selected for optimal performance (e.g., sites determined to be particularly accessible for probe hybridization) as a target in the INVADER assay.

RNAs that are generally present in predicable or invariant amounts in test samples (housekeeping controls) provide useful control targets for detection assays. These controls can be useful in several ways, including but not limited to providing confirmation of the proper function of an assay, and as a standard against which a test result for another RNA can be compared or measured to aid in interpretation of a result.

III. Detection of Allelic Expression Imbalance by Invasive Cleavage Assays

The present invention provides methods, compositions, and kits for detecting allelic expression imbalance by invasive cleavage assays, such as the INVADER assay. Allelic expression imbalance, where one allele of a particular gene is expressed at a higher level relative to the other allele, may occur when one allele is expressed to the exclusion of the other (monoallelic expression, which may occur as the result of imprinting or loss of heterozygosity) or when one is allele is simply expressed at a higher relative percent than the other allele (e.g. 60% vs. 40%). It is now becoming understood that many phenotypes and disease conditions (such as cancer) are a result of such allelic expression imbalance or even loss of expression imbalance. Due to its high level of sensitivity, invasive cleavage assays (such as the INVADER assay) may be used to detect such allelic expression imbalance or loss of expression imbalance.

When a nucleotide sequence polymorphism is present in an exon of a gene, mRNA originating from the two alleles can be distinguished by invasive cleavage assays. Messenger RNA, where genomic imprinting etc. does not arise, were previously thought to be transcribed from the two alleles in equal amounts. However, due to upstream nucleotide sequence polymorphisms or mutations affecting the control of gene expression, and differences in the 3′ terminal sequence of mRNA altering the stability of the mRNA molecule, a difference in gene expression between alleles may exist (i.e. allelic expression imbalance may exist). Therefore, detection such allelic expression imbalance (by invasive cleavage assays) can be used to detect disease conditions as well as clarify the physiological and etiological significance of nucleotide sequence polymorphisms and mutations.

Genomic imprinting (also called allelic exclusion according to parent of origin) is a mechanism of gene regulation by which only one of the parental copies of a gene is expressed. Paternal imprinting means that an allele inherited from the father is not expressed in offspring. Maternal imprinting means that an allele inherited from the mother is not expressed in offspring. Imprinted genes are the genes for which one of the parental alleles is repressed whereas the other one is transcribed and expressed. The expression of an imprinted gene may vary in different tissues or at different developmental stages. Imprinted genes may be expressed in a variety of tissue or cell types such as muscle, liver, spleen, lung, central nervous system, kidney, testis, ovary, pancreas, placenta, skin, adrenal, parathyroid, bladder, breast, pituitary, intestinal, salivary gland blood cells, lymph node and other known in art. For instance, Igf2 imprinting results in repression of the maternally-derived allele in most tissues except brain, adult liver and chondrocytes (Vu and Hoffman, Nature, 371:714-717, 1994), UBE3A (ubiquitin protein ligase 3) is paternally repressed exclusively in brain, KCNQ1 is paternally repressed in most tissues but is not imprinted in heart and WT1 (Wilms' tumor gene) is paternally repressed in cells of placenta and brain but not in kidney.

Genes may be imprinted only during specific developmental stages of an organism. For example, PEG1/MEST is maternally repressed in fetal tissue but biallelically expressed in adult blood. Also, genes may be paternally or maternally repressed in a particular species (e.g. murine versus human, Killian et al., Hum. Mol. Genet., 10:1721-1728, 2001). Loss of imprinting or LOI is said to occur when the normally silenced allele of an imprinted gene is activated. Both alleles of a gene that is usually imprinted may be expressed (e.g. at about equal levels).

Degree of allelic imbalance refers to the differential expression of the two alleles of a gene. RNA is typically transcribed from maternal and paternal genes equally (i.e. 50/50). Invasive cleavage assays maybe be used to determine a signal from both alleles (e.g. by detecting the RNA expressed for each allele), where these signals are compared to determine the degree of allelic imbalance that may exist (e.g. 70/30, 80/20, 45/55). The degree of allelic imbalance may indicate the cause of a disease as well as the severity.

Imprinting is an example where either the maternal or paternal allele is transcribed and is believed to generally correspond to a 100/0 or 0/100 expression ratio. If the gene detected is known to be expressed in an imprinted fashion (i.e. wild type has this gene imprinted in this tissue type), one may identify a disease condition by determining that both alleles are expressed (e.g. 70/30, 60/40, or 50/50).

The diseases caused by imprinting, abnormal imprinting such as LOI, and monoallelic expression include, but are not limited to, Prader-Willi syndrome, Angelman syndrome, Beckwith-Wiedmann syndrome, Silver-Russel syndrome, cancers, sudden infant death syndrome, birth defects, mental retardation, diabetes and gestational diabetes, neurological disorders, autism, bipolar affective disorder, epilepsy, schizophrenia, Tourette syndrome and Turner syndrome.

Allelic imbalance may be used as a marker for acquired DNA changes which underlie tumor formation. The method of the invention is therefore particularly useful in cancer management, including diagnosis, pre-symptomatic disease detection (screening), molecular staging and therapy monitoring. Autosomes of normal human cells are diploid, and there are two alleles derived from the father and the mother. Where the two alleles have differing sequences and are polymorphic (e.g. at a particular SNP), the gene is said to be heterozygous. The two alleles can be distinguished by this polymorphism. In cancer cells, where all or part of a chromosome is deleted, and one allele deriving from either the father or the mother has been lost, the heterozygosity that can be seen in the DNA of normal cells, cannot be found in cancer cells (called loss of heterozygosity or LOH). LOH at chromosome sites where tumor-suppressors such as p53 and APC gene are present has been recognized with high frequency in various cancers. The high frequency of cancer is resulted from the inability to suppress cell “canceration” due to the non-existence of the corresponding normal gene.

A preferred target region of interest is the APC gene (adenomatous polyposis coli gene) located on chromosome 5q (5q21), a tumor suppressor gene which has been strongly implicated in the development of colorectal cancer. Other preferred regions of interest are the DCC gene (deleted in colorectal cancer gene) located on chromosome 18q; the tumour suppressor gene p53 located on chromosome 17p (17p13); the mannose 6-phosphate/insulin-like growth factor 2 receptor tumour suppressor gene located on chromosome 6q (6q26-27); and the tumor suppressor gene p16 located on chromosome 9p (9p21). Table 1 provides a non-comprehensive list of tumor suppressor genes, their chromosomal locations and types of tumors associated with allelic imbalance of these genes. Mutations within these genes or at these chromosomal locations have been well documented. Allelic imbalance amongst these and other tumor suppressor genes can be detected using invasive cleavage assays, such as the INVADER assay.

TABLE 1

| Tumor | Chromosomal | |
|---|---|---|
| Suppressor Gene | Location | Tumor Types Observed |
| P53 | 17p13 | brain tumors, sarcomas, |
| | | leukemia, breast cancer |
| APC | 5q21 | colon cancer |
| BRCA1 | 17q21 | breast and ovarian cancer |
| BRCA2 | 13q12.3 | breast and ovarian cancer |
| NF1 | 17q11.2 | neurofibromas, gliomas, sarcomas |
| NF2 | 22q12.2 | Schwann cell tumours, astrocytomas, |
| | | meningiomas, ependymonas |
| DPC4 (Smad4) | 18q21.1 | pancreatic carcinoma, colon cancer |
| TSC1 | 9q34 | facial angiofibromas |
| TSC2 | 16 | benign growths (hamartomas) in |
| | | many tissues, astrocytomas, |
| | | rhabdomyosarcomas |
| MEN1 | 11q13 | parathyroid and pituitary adenomas, |
| | | islet cell tumours, |
| RB1 | 13q14 | retinoblastoma, osteogenic sarcoma |
| WT1 | 11p13 | pediatric kidney cancer |
| MSH2 | 2p16 | colon cancer |
| MLH1 | 3p21 | colon cancer |
| VHL | 3p26-p25 | renal cancers, hemangioblastomas, |
| | | pheochromocytoma |
| CDKN2A | 9p21 | melanoma, pancreatic cancer, others |
| PTCH | 9q22.3 | basal cell skin cancer |
| PTEN | 10q | prostatic cancer |

Another cause of allelic imbalance is amplification, particularly of oncogenes. Amplification represents one of the major molecular pathways through which the oncogenic potential of proto-oncogenes is activated during tumourigenesis (see, e.g., Schwab. BioEssays. 20:473-79, 1998, herein incorporated by reference). The following are examples of proto-oncogenes that are often amplified resulting in allelic imbalance, and thus (provided they contain a marker of heterozygosity), are detectable according to the method of this invention: MYC, ABL, RAS_{K}, RAS_{W}, MYB, ERBA, ERBB2 (also known as HER2 or NEU), MYCN and MYCL (see, e.g., Schwab & Amler. Genes Chromosom. Cancer. 1:181-193, 1990; and Schwab. BioEssays. 20:473-479, 1998).

References that describe allelic imbalance, and loss of imprinting, as well as exemplary targets and methods include the following: U.S. Pat. No. 6,586,181; US Pat. Pub. 2003/0232353; US Pat. Pub. 2003/0082616; and US Pat. Pub. 2003/0009292; all of which are herein incorporated by reference in their entireties. The present invention provides methods of detecting allelic imbalance, or loss of imprinting, using invasive cleavage assays, such as the INVADER assay.

In certain preferred embodiments, the methods of the present invention employ the INVADER assay to monitor imbalanced expression of SNP-containing alleles as described in the following exemplary embodiment. DNA and RNA may be purified from the same source of blood, or other bodily fluid or tissue, and assayed using a pair of INVADER allele specific SNP assays. In this way, the representation of each allele in the genome could be compared against the representation of each allele in the population of RNA transcripts in the experimental subject material.

First genomic DNA and mRNA can be prepared from the same biological source (e.g. blood, tumor biopsy, etc.) from a single biological specimen of interest, or from an animal or human patient. The genomic DNA can then be interrogated by a pair of INVADER SNP assays for any genetic locus of interest to determine whether the patient carried two copies of a wildtype allele (so-called “wt”), two copies of a mutant allele (so-called “mut”), or one copy of each (so-called “het”). The genomic DNA could be subjected to the INVADER assay with or without pre-amplification by PCR or another method. In certain preferred embodiments, where PCR is used, one or both of the PCR primers can also serve as the upstream oligo (INVADER oligo) rather than providing a separate upstream oligo for the INVADER assay.

The mRNAs can then be converted into cDNAs by reverse transcription. These cDNAs could then be subjected the same INVADER SNP assay used on the genomic DNA described above. This determines whether both or a single copy of the allele was being transcribed into mRNA, and the relative amount of transcript produced from each allele. For example, this study reveals that while a patient possessed “het” genomic DNA, only one allele, either “wt” or “mut” was being expressed transcriptionally.

In certain embodiments, if the genomic DNA is to be pre-amplified by PCR or other means, that the amplification means and the INVADER assay can be performed in a single tube. In particular embodiments, the sample mRNA can be converted to cDNA by reverse transcriptase and then interrogated by the INVADER assay in a single tube. In additional embodiments, the throughput of this system is increased to interrogate several loci by multiple INVADER assays in a single tube. These several loci are amplified simultaneously by multiplex PCR or other amplification means, and then simultaneously interrogated by several multiplex INVADER assays in a single tube.

All publications and patents mentioned in the above specification are herein incorporated by reference. Various modifications and variations of the described articles, devices, methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method of detecting the presence or absence of allelic expression imbalance from a heterozygous gene locus, comprising;
a) providing a sample comprising a population of target nucleic acid sequences, wherein said target nucleic acid sequences comprise: i) mRNA transcripts produced from said heterozygous gene locus, ii) cDNA products produced from said mRNA transcripts; or iii) amplified products produced from said cDNA products;
b) contacting said sample with invasive cleavage assays under conditions such that a quantitative signal for a first allele and a second allele in said population of target nucleic acid sequences is determined; and
c) comparing said quantitative signal for said first and second alleles to determine the presence or absence of allelic expression imbalance from said heterozygous gene locus in said sample.

2. The method of claim 1, wherein said invasive cleavage assays comprise first and second oligonucleotides, wherein said first and second oligonucleotides are configured to form invasive cleavage structures with said target nucleic acid sequences.

3. The method of claim 2, wherein said first oligonucleotides comprise a 5′ portion and a 3′ portion, wherein said 3′ portion is configured to hybridize to said target nucleic acid sequences, and wherein said 5′ portion is configured to not hybridize to said target nucleic acid sequences.

4. The method of claim 2, wherein said second oligonucleotides comprise a 5′ portion and a 3′ portion, wherein said 5′ portion is configured to hybridize to said target nucleic acid sequences, and wherein said 3′ portion is configured to not hybridize to said target nucleic acid sequences.

5. The method of claim 1, wherein said sample is a biological sample from a subject.

6. The method of claim 5, further comprising step d) identifying said subject as having a particular condition based on the presence or absence of allelic expression imbalance from said heterzygous gene locus in said sample.

7. The method of claim 6, wherein said particular condition is selected from the group consisting of: breast cancer, brain cancer, pancreatic cancer, loss of heterozygosity, loss of imprinting, and proper imprinting.

8. The method of claim 1, wherein said quantitative signal from said first allele is at least two percent greater than said quantitative signal from said second allele, and wherein said comparing determines the presence of allelic expression imbalance from said heterozygous gene locus in said sample.

9. The method of claim 1, wherein said invasive cleavage assays are configured to detect single nucleotide polymorphisms in said target nucleic acid sequences in order to generate said quantitative signal for said first allele and said second allele.

10. A method of detecting the presence or absence of allelic expression imbalance from a heterozygous gene locus, comprising;
a) providing a sample comprising;
i) a first nucleic acid population comprising first target nucleic acid molecules selected from: i) genomic DNA molecules comprising said heterozygous gene locus, or ii) a first amplified product produced from said genomic DNA molecules; and
ii) a second nucleic acid population comprising second target nucleic acid molecules selected from: i) mRNA transcripts produced from said heterozygous gene locus, ii) cDNA products produced from said mRNA transcripts; or iii) a second amplified product produced from said cDNA products;
b) contacting said sample with invasive cleavage assays under conditions such that a quantitative signal for a first allele and a second allele in said first and in said second nucleic acid populations is determined; and
c) comparing said quantitative signal for said first and second alleles in said second nucleic acid population to each other and to said quantitative signal for said first and second alleles in said first nucleic acid population to determine the presence or absence of allelic expression imbalance from said heterozygous gene locus in said sample.

11. The method of claim 10, wherein said invasive cleavage assays comprise first and second oligonucleotides, wherein said first and second oligonucleotides are configured to form invasive cleavage structures with said target nucleic acid sequences.

12. The method of claim 11, wherein said first oligonucleotides comprise a 5′ portion and a 3′ portion, wherein said 3′ portion is configured to hybridize to said target nucleic acid sequences, and wherein said 5′ portion is configured to not hybridize to said target nucleic acid sequences.

13. The method of claim 11, wherein said second oligonucleotides comprise a 5′ portion and a 3′ portion, wherein said 5′ portion is configured to hybridize to said target nucleic acid sequences, and wherein said 3′ portion is configured to not hybridize to said target nucleic acid sequences.

14. The method of claim 10, wherein said sample is a biological sample from a subject.

15. The method of claim 14, further comprising step d) identifying said subject as having a particular condition based on the presence or absence of allelic expression imbalance from said heterozygous gene locus in said sample.

16. The method of claim 15, wherein said particular condition is selected from the group consisting of: breast cancer, brain cancer, pancreatic cancer, loss of heterozygosity, loss of imprinting, and proper imprinting.

17. The method of claim 10, wherein said invasive cleavage assays are configured to detect single nucleotide polymorphisms in order to generate said quantitative signal for said first allele and said second allele in said first and second nucleic acid populations.

18. A kit comprising first and second invasive cleavage assays configured for detecting the presence or absence of allelic expression imbalance from a heterozygous gene locus, wherein said first invasive cleavage assay is configured to generate a quantitative signal for a first allele of said heterozygous gene locus, and said second invasive cleavage assay is configured to generate a quantitative signal for a second allele of said heterozygous gene locus.

19. The kit of claim 18, wherein said first and second invasive cleavage assays comprise first and second oligonucleotides, wherein said first and second oligonucleotides are configured to form invasive cleavage structures with target nucleic acid sequences.

20. The kit of claim 19, wherein said first oligonucleotides comprise a 5′ portion and a 3′ portion, wherein said 3′ portion is configured to hybridize to said target nucleic acid sequences, and wherein said 5′ portion is configured to not hybridize to said target nucleic acid sequences.
